(19) United States
(12) Patent Application Publication
(10) Pub. No.: US 2021/0236449 A1
(43) Pub. Date: Aug. 5, 2021

(54) USE OF CHLOROGENIC ACID IN PREPARING DRUG FOR PREVENTING OR BLOCKING BRAIN AND/OR BONE METASTASES OF LUNG CANCER

(71) Applicant: SICHUAN JIUZHANG BIOLOGICAL SCIENCE AND TECHNOLOGY CO., LTD, Chengdu, Sichuan (CN)

(72) Inventors: Jie ZHANG, Chengdu, Sichuan (CN); Xiaoguang CHEN, Chengdu, Sichuan (CN); Wang HUANG, Chengdu, Sichuan (CN)

(21) Appl. No.: 15/733,748

(22) PCT Filed: Apr. 15, 2019
(86) PCT No.: PCT/CN2019/082765

§ 371 Date: Oct. 15, 2020

(30) Foreign Application Priority Data
Apr. 16, 2018 (CN) 201810339463.9

(51) Int. Cl. A61K 31/216; A61P 35/04

(52) U.S. Cl. CPC A61K 31/216; A61P 35/04

(57) ABSTRACT

Chlorogenic are used in preparing a drug for preventing or blocking brain and/or bone metastases of lung cancer. The chlorogenic acid can effectively prevent or block metastasis of tumor cells of a patient with lung cancer, such as small cell lung cancer, squamous cell lung cancer, and lung adenocarcinoma.

### TECHNICAL FIELD

The present invention belongs to the field of biomedicine, and specifically relates to the use of chlorogenic acid in preparation of drugs for preventing and blocking brain metastases and/or bone metastases of lung cancer.

### BACKGROUND ART

Lung cancer, with high incidence and mortality, is one of the malignant diseases originating from the bronchial epithelium in humans. Among them, non-small cell lung cancer (NSCLC) accounts for 80%, with 5-year survival rate of 15%, while small cell lung cancer (SCLC) accounts for 15%-20%, and its malignant degree is higher. SCLC has deteriorated or even metastasized when it is discovered, and lung cancer metastasis is the main cause of treatment failure and patient death. Metastasis to various organs can occur in the late stage of lung cancer, which can cause corresponding symptoms, and often bring great pain and even life-threatening to patients. The most common clinical metastases include brain and bone metastasis of lung cancer.

Brain metastasis of lung cancer is a common metastatic site of advanced NSCLC, accounting for 30%-55% of patients. Patients with lung cancer have unexplained headaches, vomiting, visual disturbances, and changes in personality and temper, which may be caused by internal hypertension or brain nerve damage due to metastasis of lung cancer to the brain. That is one of the main causes of death in patients with small cell lung cancer.

Bone metastasis occurs in 30%-40% of patients with advanced lung cancer, and bone metastasis is not the direct cause of life-threatening patients with lung cancer, but if lung cancer metastasizes to the body's load-bearing bones such as cervical, thoracic, lumbar and other parts, serious consequences including paralysis can be caused. Bone pain and a series of bone-related events in patients are important factors that reduce the life quality of patients and affect their survival. Tumor metastasis is a series of complex processes involving multiple steps, multiple stages, multiple pathways, and multiple genetic changes, including that tumor cells fall off from the primary tumor, invade blood vessels or lymphatic vessels, migrate and adhere to appropriate sites to induce tumor angiogenesis, resist host's anti-tumor immunity, and eventually form metastases in the distance. Studies have shown that in the infiltration and metastasis process of tumor cells, not only a variety of genes are involved, but also some related factors, enzymes and proteins work together.

Che et al. have first proposed that the enhancement of YWHAZ is related to the malignant degree of lung cancer. They have found that YWHAZ gene is a potential regulator of β-catenin, prevents β-catenin from ubiquitination and degradation, promotes the subsequent EMT phenotype, and improves the invasion ability of cancer cells.

Jan et al. pointed out that adenylate kinase 4 (AK-4) gene can enhance the metastasis ability of human lung cancer, and reducing the expression of AK4 can lower the invasion ability of cancer cell lines, while high expression of AK4 can significantly improve the invasion ability of cancer cells in in vivo and in vitro experiments. Studies have shown that activating transcription factor 3 (ATF3) is the central target of AK4 regulation, and the patients with high expression of AK4 and low expression of ATF3 have a poor prognosis. AK4 improves the malignant progression and recurrence of tumors in an ATF-3 dependent manner. AK4 can inhibit the expression of ATF3 and activate the downstream effector MMP2, which promotes the invasion-metastasis cascade of lung cancer and enhances the metastasis of lung cancer cells.

Li et al. have indicated that interleukin 17 (IL-17) can directly improve the metastatic ability of lung cancer, which is partly due to IL-6-Stat3 pathway.

Kuramoto et al. have recently reported that D114-Notch signaling pathway plays an important role in the process of liver metastasis of small cell lung cancer, which is achieved by down-regulating the activity of Notchl in NF-KB signaling pathway.

Song et al. have found that down-regulating the expression of CAV-1 inhibited the proliferation of NCI-H460 cell lines, but significantly improved its in vitro metastatic ability. This phenotype is related to the inhibition of E-cadherin pathway and the down-regulation of cyclin D1 and PNCA. Tumor metastasis is the main reason for the failure of malignant tumor treatment and the death of patients. The prior treatment methods still cannot effectively improve the survival rate of patients; therefore, the research strategy of anti-lung cancer metastasis provides new directions and ideas for lung cancer treatment.

### Content of the Invention

The present invention provides the new use of chlorogenic acid.

The present invention provides the use of chlorogenic acid in preparation of drugs for preventing and blocking lung cancer metastasis.

Wherein, said lung cancer metastasis is brain metastasis and/or bone metastasis of lung cancer.

Wherein, said lung cancer includes small cell lung cancer or non-small cell lung cancer.

Wherein, said non-small cell lung cancer is lung squamous cell carcinoma or lung adenocarcinoma.

Wherein, said lung cancers are those moderately differentiated or poorly differentiated.

Wherein, said medicament is a commonly used oral or injectable preparation in pharmacy prepared by using chlorogenic acid as an active ingredient, with addition of pharmaceutically acceptable excipients.

Wherein, each preparation unit in the pharmaceutical preparation contains 1-3000 mg of chlorogenic acid.

Wherein, the dosage of chlorogenic acid in the pharmaceutical preparation is 1-100 mg/kg.

Wherein, the medicament is oral preparation or injection.

Wherein, said lung cancer metastasis is lymph node metastasis of lung cancer, and preferably, the content of chlorogenic acid in the unit preparation of the drug is 155.16 mg.

The present invention proves that chlorogenic acid can effectively prevent and block lung cancer metastasis, especially the effect on brain metastasis and bone metastasis of lung cancer is clear, and thus can effectively solve the problem of cancer cell metastasis in the treatment of lung cancer with traditional chemotherapy drugs, with a good clinical application prospect.

Obviously, based on above content of the present invention, according to the common technical knowledge and the conventional means in the field, without department from above basic technical spirits, other various modifications, alternations or changes can further be made.

By following specific examples of said embodiments, above content of the present invention is further illustrated. But it should not be construed that the scope of above subject of the present invention is limited to following examples. The techniques realized based on above content of the present invention are all within the scope of the present invention.

### DESCRIPTION OF FIGURES

FIG. 1 In vivo imaging of the middle-dose group of chlorogenic acid (CHA) and model control group (Control).

FIG. 2 Photographs of bone tissues in chlorogenic acid middle-dose group and model control group.

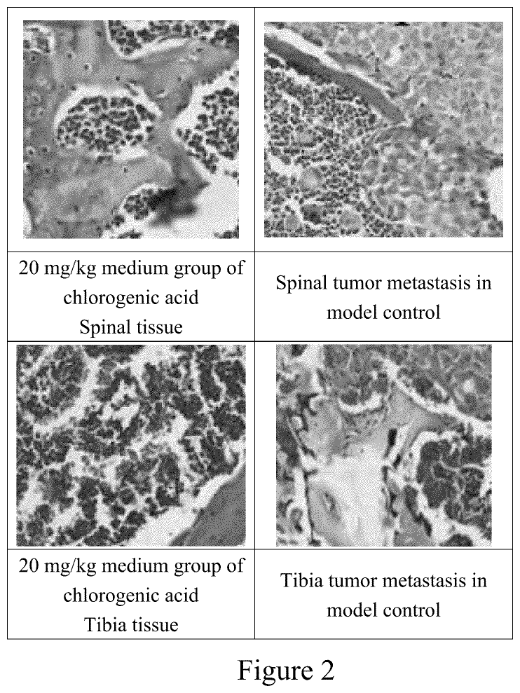

FIG. 3 Photographs of brain tissues in chlorogenic acid middle-dose group and model control group.

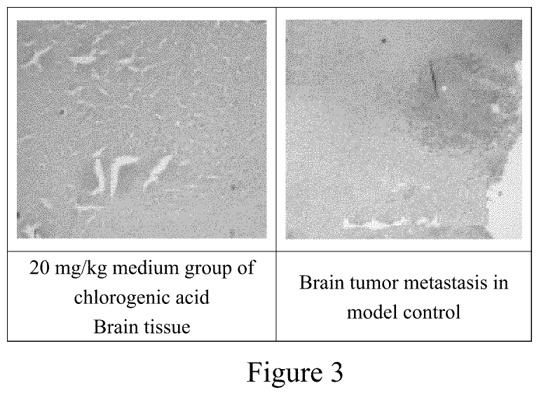

### EXAMPLES

### Experimental Example 1

### Inhibitory Effect of Chlorogenic Acid on Metastasis in LA795 Lung Cancer Mice

### 1.1 Materials

(1) Animals: T739 mice, half male and half female, weighing 18-22 g, are provided by the Experimental Animal Center of Sichuan University.

(2) Lung cancer cell lines: LA795, provided by Wuhan Procell Life Technology Co., Ltd.; Fetal bovine serum, provided by Hangzhou Sijiqing Bioengineering Materials Co., Ltd.; plasmid EGFP-C1, provided by Clontech; Liposome transfection reagent Lipofectamin 2000, provided by Invitrogen.

### 1.2 Apparatus

Nuance multi-spectral fluorescence inverted microscope, Maestro multi-spectral live animal imaging system (CRi, USA), FACSArray flow cytometer (BD, USA).

### 1.3 Cell Culture and Transfection

LA795 cells were inoculated in a 6-well culture plate at 1×10⁷ cells/mL, using DMEM complete medium containing 10% fetal bovine serum, and cultured in a 37° C., 5% CO₂ incubator. When the cells were about 70% fused, liposome transfection reagent lipid Lipofectamin2000 was used and LA795 cells were transfected into plasmid EGFP-C1. 48 h after transfection, cells were passed and screened by G 418, then anti-G418 fluorescent positive clones appeared after about 4 weeks. Clones were observed under fluorescent inverted microscope, and one resistant fluorescent cell clone was randomly picked and transferred to 96-well plate for culture. After stepwise expansion, cells were routinely cultured and passaged. 400 μg/ml G418 was used to maintain screening and obtain expanded EGFP-LA795 cells. When EGFP-LA795 cells are 80%-90% fused in the logarithmic growth phase, they are digested with 0.25% trypsin, then centrifuged, and the cells are resuspended in DMEM to prepare a cell suspension for seeding (cell concentration 1×10⁷ cells/mL). EGFP-LA795 cells grown on the wall were trypsinized to prepare a single cell suspension, and then diluted appropriately. The cell suspension was mixed with 0.4% trypan blue solution and counted under the microscope. The survival rate of EGFP-LA795 cells was >90%, and the positive clones were picked out under an inverted fluorescence microscope, followed by culture and amplification, to obtain LA795 cells with green fluorescence.

### 1.4 Model Building

0.2 ml of EGFP-LA795 cell suspension at 1.0×10⁷ cells/ml was inoculated into the armpit of mice, and then the mice were randomly divided into groups, 10 mice for each group, including the high-dose chlorogenic acid group, the middle-dose chlorogenic acid group, and the low-dose chlorogenic acid group and the model control group. Chlorogenic acid was administered by intraperitoneal injection, 40 mg/kg for the high-dose group, 20 mg/kg for the middle-dose group, 10 mg/kg for the low-dose group, and the administration volume was 1.0 ml; the model control group was given the same volume of normal saline by intraperitoneal injection, once a day for 20 days. In vivo imaging observations were performed on days 0, 10, and 20, respectively. After the test, the mice were sacrificed by cervical dislocation, and peripheral lymph nodes, axial bones, and long bones of the limbs were taken to observe and count metastases.

### 1.5 In Vivo Imaging of Mice

10% chloral hydrate was intraperitoneally injected at 4 ml/kg for anesthetization, and mice were put on the black rubber pad prepared for the in vivo imager. The irradiation conditions were GFP, the emission wavelength was 520 nm, the excitation wavelength was 480 nm, and the exposure time was 5 ms. The volume changes and metastasis of the tumor were observed.

### 1.6 Metastasis Count

Surrounding lymph nodes were taken out, and 8 μm conventional and frozen sections were obtained, and routinely stained with hematoxylin-eosin (HE) every other one, to observe the tumor metastasis under fluorescence microscope and light microscope.

Axial bones (such as ribs, spine, ilium, scapula) and long bones of the limbs of mice were chosen, cut into 0.5 cm×0.3 cm×0.3 cm bone pieces, and fixed with frozen 4% paraformaldehyde solution at 5° C. for 12 h-24 h; the bones were rinsed with phosphate buffer and decalcified with EDTA until the bone slices were completely decalcified. After decalcification, the bone slices were rinsed with distilled water for 20 minutes, subjected to conventional dehydration treatment, embedded in paraffin, sectioned (thickness 4 μm), and then stained with hematoxylin-eosin for microscopic examination.

The brain tissue of mice was taken out by craniotomy, and a few of 1 mm³ tissue pieces were randomly cut off, rinsed and fixed overnight, dehydrated with alcohol and acetone, and embedded in epoxy resin 618. The ultrathin sections were stained with lead and examined under a microscope.

### 1.7 Experimental Results

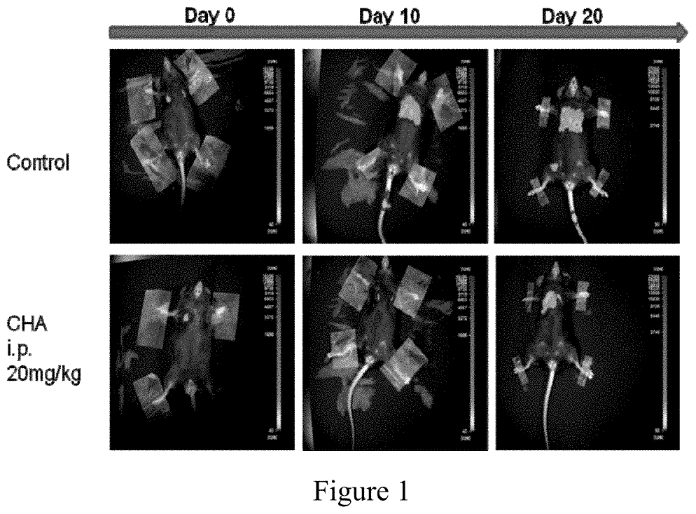

(1) In vivo imaging of mice (FIG. 1).

(2) In vivo invasion and metastasis conditions of EGFP-LA795 cells (see Table 1)

TABLE 1
Effects of chlorogenic acid on in vivo invasion and
metastasis conditions of EGFP-LA795 cells (n = 10)

| Experimental | Abdominal | Local lymph node | Bloody | Axial bone | Limb bone | Brain |
|---|---|---|---|---|---|---|
| groups | metastasis | metastasis | ascites | metastasis | metastasis | metastasis |
| Chlorogenic acid | 1/10* | 1/10* | 0/10* | 0/10* | 0/10* | 0/10* |
| high dose group | | | | | | |
| Chlorogenic acid | 0/10* | 0/10* | 0/10* | 0/10* | 0/10* | 0/10* |
| medium dose group | | | | | | |
| Chlorogenic acid | 2/10* | 1/10* | 0/10* | 0/10* | 0/10* | 0/10* |
| low dose group | | | | | | |
| Model control group | 9/10 | 10/10 | 8/10 | 9/10 | 6/10 | 5/10 |

Note:
*indicates p < 0.05 compared with model control group

The experimental results showed that the number of metastatic foci in mice of the low, medium, and high dose chlorogenic acid test groups according to the present invention was significantly reduced, which was significantly different from the model control group. Thus, chlorogenic acid could effectively prevent and inhibit the metastasis of LA795 lung cancer cells in mice, and especially the preventive and inhibitory effects of 20 mg/kg medium-dose group of chlorogenic acid were very significant. No metastasis was found, and thus the medium-dose group could effectively prevent or inhibit the metastasis and spread of tumors. Among them, the inhibitory effect on brain and bone metastasis was particularly good.

Experimental results confirmed that chlorogenic acid could effectively inhibit brain and bone metastasis of lung cancer, and the effect was very good.

The experimental results also confirmed that chlorogenic acid could effectively inhibit the lymph node metastasis of lung cancer. Among them, when the dose was 20 mg/kg (medium dose), the effect was particularly good, and by exchanging, the human dose was 2.21 mg/kg. The exchange process was as follows:

Human dose: 20 mg/kg mouse dose*0.02 kg mouse weight*387.9 body surface area coefficient/70 kg adult body weight=2.21 mg/kg.

Based on the average body weight 70 kg, the content of chlorogenic acid in the unit preparation was 155.16 mg.

In summary, using the dose of chlorogenic acid and the specific method according to the present invention to treat patients with lung cancer had obvious therapeutic effects on preventing and blocking brain metastasis and/or bone metastasis of lung cancer. At the same time, it could alleviate the clinical symptoms of patients, improve the life quality of patients, and effectively prolong the life of patients, with a good clinical application prospect.

1. The use of chlorogenic acid in preparation of drugs for preventing and blocking lung cancer metastasis.

2. The use according to claim 1, characterized in that said lung cancer metastasis is brain metastasis and/or bone metastasis of lung cancer.

3. The use according to claim 1, characterized in that said lung cancer includes small cell lung cancer or non-small cell lung cancer.

4. The use according to claim 3, characterized in that said non-small cell lung cancer is lung squamous cell carcinoma or lung adenocarcinoma.

5. The use according to claim 1, characterized in that said lung cancers are those moderately differentiated or poorly differentiated.

6. The drug according to claim 1, characterized in that said medicament is a commonly used oral or injectable preparation in pharmacy prepared by using chlorogenic acid as an active ingredient, with addition of pharmaceutically acceptable excipients.

7. The use according to claim 6, characterized in that each preparation unit in the pharmaceutical preparation contains 1-3000 mg of chlorogenic acid.

8. The use according to claim 7, characterized in that the dosage of chlorogenic acid in the pharmaceutical preparation is 1-100 mg/kg.

9. The use according to claim 6, characterized in that the medicament is oral preparation or injection.

10. The use according to claim 1, characterized in that said lung cancer metastasis is lymph node metastasis of lung cancer, and preferably, the content of chlorogenic acid in the unit preparation of the drug is 60-120 mg.
